(19) United States
(12) Patent Application Publication
(10) Pub. No.: US 2003/0207263 A1
(43) Pub. Date: Nov. 6, 2003

(54) Method of screening therapeutic agents

(76) Inventors: Sylviane Gabrielle Nadine Dennler, Les Ulis (FR); Jean Michel Gauthier, Les Ulis (FR); Staphane Huet, Les Ulis (FR)

Correspondence Address: NIXON & VANDERHYE, PC, 1100 N GLEBE ROAD, 8TH FLOOR, ARLINGTON, VA, 22201-4714, US

(21) Appl. No.: 09/601,534

(22) PCT Filed: Feb. 4, 1999
(86) PCT No.: PCT/EP99/00664

(30) Foreign Application Priority Data
Feb. 6, 1998 (GB) 9802475.5

(51) Int. Cl. C12Q 1/68; G01N 33/574

(52) U.S. Cl. 435/6; 435/7.23

(57) ABSTRACT

The invention relates to a method for screening therapeutic agents for use in combating diseases associated with gene regulation by one or more Smad proteins and TGF&bgr; or activin, said method comprising detecting or assaying the extent or result of transcriptional activity or binding in the presence of said agent between a Smad protein or a DNA binding fragment thereof and a double strand oligonucleotide comprising the sequence 5′ WXYCAGACZ 3′ or a functional equivalent thereof, wherein in said nucleotide sequence W represents A or G, X represents G or T, Y represents C, A, G or T and Z represents A or C. Also claimed are therapeutic agents identified by such a method and their use in combating diseases associated with abnormal expression of Smad-mediated TGF&bgr;-induced genes.

The present invention relates to a nucleotide sequence, in particular a transcriptional regulatory sequence which confers TGFβ and activin induction and which binds Smad proteins, and to uses of the sequence for example in screening agents for utility in combating diseases associated with abnormal expression of Smad-mediated TGFβ-induced genes.

Transforming growth factor β (TGFβ) belongs to a family of cytokines, including activin and Bone Morphogenetic Proteins, which are synthesised by many cell types and have a variety of cellular and biological effects, including control of proliferation, differentiation, migration, immunity and regulation of the turnover of the extracellular matrix. In many of these effects TFGβ, as exemplified by TGFβ-1, acts as a transcription activator. Several promoters are known to be induced by TGFβ, including Plasminogen Activator Inhibitor-type 1 (PAI-1), α2 (I) procollagen, TGFβ-1 itself, germ line lgα constant region, the cyclin-dependent-kinase (CDK) inhibitors p21 and p15.

Members of the Smad family of proteins play a vital role in mediating TGFβ and activin transcriptional activation via a mechanism which is not entirely elucidated. The amino-terminal part of the Drosophila MAD ortholog protein has been shown to bind to an enhancer of the vestigial gene that is important for transcriptional regulation (Kim et al. Nature, 1997, 388, 304-308). The Xenopus Smad2 and Smad4 proteins are components of a protein complex named Activin-Response Factor (ARF) that contains also the FAST-1 transcription factor. ARF ability to bind to the activin-induced Xenopus Mix.2 promoter is conferred by FAST-1 and Smad2/Smad4 are proposed to act as co-activators (Chen et al. Nature, 1996, 383, 691-696; Chen et al. Nature, 1997, 389, 85-89). Of those Smad proteins involved in TGFβ signalling, Smad 6 and 7 are known to act as inhibitors of TGFβ signalling pathway, Smad 2 and 3 are known to mediate the TGFβ signalling pathway and Smad 4 is known to form heteroligomers with at least Smad 2 and 3 (Heldin et al. Nature, 1997, 390, 465-471). Smad 4 has been shown to bind a DNA sequence of an artificial construct but this binding activity does not confer TGFβ-dependent transcriptional activation (Yingling et al. Mol. Cell. Biol., 1997, 17, 7019-7028).

We have now shown the existence of a complex including two Smad proteins, Smad 3 and Smad 4, and DNA and demonstrated that Smad 3, Smad 4 are DNA binding proteins. We have also demonstrated that Smad2 spliced in exon 3 is a DNA binding protein. Furthermore, we have identified the Smad 3/4-binding sequence within a TGFβ-responsive promoter and shown that binding of Smad 3/4 is essential for the TGFβ induced transcriptional effect.

A number of disease states are known to be associated with variations in expression of genes which are controlled by TGFβ, including fibrotic disorders, abnormal wound healing, abnormal bone formation, cancer development, haematopoiesis, neuroprotection and immune and inflammatory disorders. The PAI-1 gene is one of the genes activated by TGFβ the most studied. PAI-1 protein is produced by several cell types including endothelial cells, fibroblasts, epithelial cells and liver parenchymal cells. It indirectly controls the activity of the serine protease plasmin by virtue of its inhibitory action on urokinase (U-PA) and tissue plasminogen activator (t-PA), each of which catalyse the formation of plasmin from plasminogen.

Plasmin plays an important role in formation and maintenance of the extracellular matrix both directly, by digesting matrix components and indirectly, by its ability to activate latent forms of matrix degrading enzymes. The major role of plasmin is in removing fibrin clots. Thus plasmin has dual specificity towards the vasculature (ie. fibrin) and the matrix. Since plasmin levels are controlled by PAI-1, PAI-1 thus has an important role in influencing the fibrinolytic balance and controlling the amount of fibrotic lesions. The ability to modulate matrix deposit is important therapeutically in a number of indications including wound healing, hypertrophic scars, keloids, scleroclerma, hepatic and biliary fibrosis, lung fibrosis, kidney fibrosis, cardiac fibrosis and post surgical adhesions (Franklin. Int. J. Biochem. Cell Biol., 1997, 29, 78-89). At present, there is no therapy for fibrosis.

Our findings that Smad3, Smad4 and Smad2 spliced in exon 3 are DNA binding proteins which bind to TGFβ activated promoters such as PAI-1 paves the way for the development of new strategies for combating diseases associated with Smad-mediated TGFβ gene regulation by modulating the binding or the transcriptional activity of Smad3 or Smad4 or Smad2 spliced in exon 3 (or indeed any Smad3 or Smad4 containing protein complex), to its recognition sequence, and to methods of screening pharmaceutical agents capable of modulating the expression of TGFβ-regulated genes for use in therapy by affecting the degree of Smad containing complex (i.e. Smad3 and Smad4 and Smad2 spliced in exon 3) binding to its recognition sequence or the transcriptional ability of Smad containing complex (i.e. Smad3 and Smad4 and Smad2 spliced in exon 3) bound to its recognition sequence in promoters of genes thus affected.

Thus, according to one aspect, the present invention provides methods for screening agents for use in combating diseases associated with gene regulation by Smad and TGFβ or activin, said method comprising detecting or assaying the extent or result of transcriptional activity or binding in the presence of said agent between a Smad protein or a DNA binding fragment thereof and a double strand oligonucleotide comprising the sequence 5′ WXYCAGACZ 3′ or a functional equivalent thereof, wherein in said nucleotide sequence W represents A or G, X represents G or T, Y represents C, A, G or T and Z represents A or C.

We have named this sequence the CAGA box. As used herein, the term CAGA box is used to refer not only to the sequence which we have identified in the PAI-1 promoter but also to any sequence functionally equivalent to such a sequence i.e. to any nucleotide sequence capable of binding an Smad protein either individually or as part of a complex of Smad proteins whereby such binding is a necessary step for TGFβ and activin regulation of genes under the control of such functionally equivalent sequence.

As used herein, the term ‘screening’ includes any method or assay whereby the action of an agent capable of modulating, affecting, influencing or interfering with the binding between a Smad protein and the CAGA box or the transcriptional ability of a Smad protein bound to the CAGA box is investigated, and includes binding assays in which a single agent or compound is investigated as well as assays in which more than one compound, such as an array of compounds, or a library of compounds is tested. In the case of testing more than one agent, these tests may be either simultaneous or sequential. Such agents may act either to interfere with the binding of a Smad protein such as Smad3 or Smad4 or Smad2 spliced in exon 3 to the CAGA box sequence, i.e. to prevent wholly or partially Smad binding to the CAGA box, or they may enhance the binding between a Smad protein and the CAGA box. Such agents may act also to modulate the transcriptional activity of a Smad protein bound to the CAGA box sequence such as Smad3 or Smad4 or Smad2 spliced in exon 3, i.e. to decrease the transcriptional activity of a Smad containing complex bound to the CAGA box, or they may enhance the transcriptional activity of a Smad containing complex bound to the CAGA box . The methods of detection and assay include any quantitative, qualitative or semiquantitative assessment of whether there is any binding or transcriptional activity, and of the effect of the agent being tested. Preferably for screening agents of therapeutic benefit in combating diseases associated with Smad3/Smad4/Smad2 spliced in exon 3/TGFβ/activin regulation, it is compounds which have a modulating effect on Smad3/Smad4/Smad2 spliced in exon 3/DNA complex formation or transcriptional activity which are investigated by the screening test.

The term ‘an Smad protein’ is used herein to refer to a protein or a protein complex having the binding characteristics of an Smad protein which binds to its receptor sequence (the CAGA box) such as Smad3 or Smad4 or Smad2 spliced in exon 3 either alone or as a protein complex, and includes DNA binding fragments of these proteins, fusion proteins containing these proteins and modifications, as well as referring to the Smad3 and Smad4 and Smad2 spliced in exon 3 proteins themselves.

In a preferred aspect, the double strand oligonucleotide comprises the sequence AG(C/A)CAGACA, which is the sequence we have identified in the PAI-1 promoter. We have identified the sequence AG(C/A)CAGACA present in three copies in the human PAI-1 promoter in regions known to mediate TGFβ transcriptional induction. This sequence, and sequences closely similar to this sequence comprising the—CAGA—motif has also been identified in other promoters and enhancers known to be inducible by TGFβ including α2(I) procollagen, the germ line lgα constant region and TGFβ1 promoter. These sequences are presented in Table 1 and are included in the term CAGA box.

TABLE 1

| Promoter | Sequence | Position |
|---|---|---|
| human PAI-1 promoter | AGCCAGACA | −730 |
| | AGACAGACA | −580 |
| | AGACAGACA | −280 |
| human TGFβ-1 gene | AGCCAGACA | +22 |
| human α2(I) collagen promoter | ATGCAGACA | −264 |
| human germ line IGα constant region | AGCCAGACC | −120 |
| | GGCCAGACA | −35 |

In one aspect, the oligonucleotide for use in the screening test of the invention comprises the CAGA box itself. The CAGA box may, however, include flanking sequences at one or both ends. Such sequences may extend the length of one strand of the CAGA box by, for example, 3 nucleotides to a total of 12 nucleotides in length, either 3 nucleotides at one end, or 2 nucleotides at one end, and one at the other, or they may extend the sequence by 6 nucleotides to a total of 15 nucleotides, with the additional bases at one end or divided between each end of the CAGA box itself, or the flanking sequences may extend one strand of the CAGA box further e.g. to a total of 20 nucleotides or more such as up to 30, 40 or 50 nucleotides. For use in the invention the oligonucleotide may comprise the CAGA box itself, or the CAGA box extended by up to 10 nucleotides, preferably up to 20 nucleotides, and preferably up to 50 nucleotides. The CAGA box, optionally with flanking regions may be repeated in the oligonucleotide for use in the invention, for example up to 50 repeats, preferably up to 20 repeats, such as up to 10 repeats. The term test oligonucleotide as used herein includes the CAGA box and all these oligonucleotides based on the CAGA box. Preferably such sequences are distinct from AP-1 binding sites.

In a preferred aspect, Y represents C, A or G.

For use in the method of the invention, the test oligonucleotides may be synthesised chemically or they may be genomic or cDNA fragments or incorporated in recombinant vectors such as those based on plasmids or bacteriophage.

In one aspect, the present invention involves comparing either the binding between a Smad protein and the test oligonucleotide or the transcriptional activity of a Smad containing protein complex bound to the test oligonucleotide, in the presence of a test agent with that in the absence of said agent.

We have shown that this TGFβ inducible CAGA box is specifically involved in Smad mediated TGFβ induction. Thus, when cloned in multiple copies upstream of the TK promoter, the CAGA box sequence has been found to confer TGFβ mediated transcriptional induction in HepG2 cells, but a mutated version of this sequence, AGCTACATA, i.e. a sequence containing three point mutations did not confer TGFβ induction. We have shown that Smad4 is essential in TGFβ mediated induction in MDA-MB4648 cells which are human epithelial cells derived from a breast cancer which are deficient for Smad4, where TGFβ had no effect on expression of a CAGA reporter construct, but induction by TGFβ was observed when this cell-line was cotransfected with an expression construct encoding for Smad4. We demonstrated the binding properties of the CAGA sequence using electrophoretic mobility-shift assays (EMSA) of HepG2 nuclear extracts in the presence of TGFβ and antibodies to different Smad proteins, showing that Smad3 and 4 were present in the TGFβ-dependent CAGA box binding complex, and using EMSA in the presence of E. coli expressed Smad proteins we demonstrated that Smad3 and Smad4 had a direct and specific DNA-binding activity. Furthermore, we have shown that the closely related Smad2 protein was not able to activate CAGA-mediated transcription. We demonstrated that the domain encoded by exon3 in the Smad2 gene prevented Smad2 from binding to the CAGA sequence and that a version of Smad2 where the domain corresponding to exon 3 is not present was able to bind to and activate transcription from the CAGA box.

Sequences similar to our CAGA box have been identified in other TGFβ inducible regions of promoters regulated by TGFβ, such as α2(I) procollagen gene, the germ line lgα2 construction region gene and TGFβ1 promoters. These sequences are presented in Table 1.

The method of screening potentially useful pharmacological agents for modulating the transcriptional ability or the binding of one or more Smad proteins alone or in a complex on the CAGA box containing sequence or a functionally equivalent sequence and ultimately modifying the expression of genes controlled by Smad-TGFβ induction may be carried out in a variety of direct or indirect ways.

In the direct type of method, the formation of a binding complex between a protein (ie. an Smad or a CAGA binding fragment thereof) and a test oligonucleotide or a CAGA containing nucieotide sequences is analysed. A variety of techniques known in the art may be utilised for this using as the protein element any Smad protein which has the ability to form complexes with a CAGA related recognition sequence, such as, for example, a mammalian Smad3 and or Smad4 or a Smad2 protein spliced in exon 3 or a CAGA box binding fragment thereof, either alone or as part of a recombinant polypeptide, which may be purified from cells or from expression systems known in the art, including procaryotic expression systems using bacteria such as E. coli or eucaryotic expression systems such as yeast or baculovirus, or in vitro expression systems for example those based on reticulocyte lysates. Such techniques are described in for example Sambrook et al., Molecular Cloning: A laboratory Manual 1989. The DNA part of the specific binding complex may comprise oligonucleotides including the test oligonucleotides which comprise the CAGA box containing recognition sequence, these oligonucleotides may be either synthesised chemically or be genomic or cDNA fragments, or be part of recombinant vectors for example those based on plasmids or bacteriophage.

Methods for screening the interaction between DNA and protein in accordance with the invention are known in the art. Thus known amounts of protein and DNA can be admixed and after complex formation has taken place, the amount of uncomplexed DNA or protein can be determined. Uncomplexed protein may be measured by various techniques which include antibody detection for example by enzyme linked immunosorbent assay (ELISA) and standard protein measuring techniques such as the Lowry, biuret or Bradford assay once the complex has been separated. Uncomplexed DNA may be determined again by a variety of techniques known in the art, for example by hybridization with a detectably labelled probe such as biotin or radioactive labels, and wherein the probes may be immobilised or in solution. The complex between polypeptide and DNA may also itself be measured using techniques known per se including footprinting, EMSA, scintillation proximity assay (SPA), biacore or biochip/DNA chip technologies.

Alternatively, the extent of polypeptide-DNA complex formation or the transcriptional ability of the polypeptide-DNA complex can be determined by virtue of the effect it has on transcription. In a method known as transcriptional screening, the invention may be used to screen agents that activate or inhibit the TGFβ or activin transduction pathway from cell membrane to the nucleus that in fine leads to CAGA box-mediated transcriptional regulation. In such an approach, the CAGA box containing oligonucleotidic sequence may be cloned in a vector such as a reporter vector for example a plasmid in operable linkage to a promoter and/or enhancer controlling a nucleotide sequence which expresses a detectable protein for example, luciferase, alkaline phosphatase, chloramphenicol acetyl transferase, β-galactosidase wherein in such a construct the level of expression of such a reporter gene can be detected after transient or stable transfection of the reporter construct into eukaryotic cells. Thus in such a transcriptional screen, the CAGA box containing nucleotidic sequence is integrated within the regulatory region of a gene whose product can be detected in an in vitro system, and the level of product expressed in transfected cells incubated in the presence of test agent (and in the presence or the absence of TGFβ or activin) is compared to that expressed in transfected cells incubated in the absence of test agent (and in the presence or the absence of TGFβ or activin).

Preferably, in the reporter vector for use in this aspect of the method, suitable expression control sequences will be provided such as translational e.g. stop, start codons, and control elements in addition to promoter/enhancer regions such as Poly-adenylation signal etc.

In a preferred aspect, the method of the invention may be used to screen agents of potential use in the therapies of diseases where unregulated expressions of genes controlled by TGFβ are known to be involved such as fibrosis, abnormal wound healing, cancer, haematopoiesis or immunity or inflammation disorders. In particular, such agents by interfering with the binding of Smad to DNA mediated by TGFβ or activin or by interfering with the transcriptional ability of Smad bound to DNA will modulate the synthesis of plasminogen activator inhibitor type 1 and thus affect plasmin levels, thereby modulating matrix formation and/or fibrinolysis.

Viewed from a further aspect, the present invention provides a kit for screening agents suitable for combating diseases associated with Smad mediated TGFβ or activin activation, said kit comprising:

a Smad protein as hereinbefore defined

TGFβ or activin

a double strand DNA molecule comprising the sequence 5′YCAGACZ3′ as hereinbefore defined, said sequence optionally being in operable linkage with a promoter sequence and coding region of a gene whose product is detectable.

The recognition of the CAGA related sequence in accordance with the invention as being necessary for TGFβ or activin transcriptional regulation by means of Smad offers a new genetic approach to therapy of those diseases, such as fibroses, abnormal wound healing, haematopoiesis or immune or inflammatory disorders. and cancer, where there is an association with TGFβ regulation of certain genes.

Thus viewed from a further aspect, the present invention comprises a method of treating a disease associated with gene regulation by means of one or more Smad proteins and TGFβ or activin, said method comprising administering a double strand oligonucleotide comprising the sequence 5′VXYCAGACZ3′ as hereinbefore defined.

In such a method, Smad proteins are sequestered by the exogenously administered DNA and thereby prevent TGFβ mediated induction of endogenous genes.

Viewed from a further aspect, the present invention provides an isolated double strand DNA molecule comprising the sequence 5′ WXYCAGACZ 3′ as hereinbefore defined. Preferably the sequence is AG(C/A)CAGACA. The invention also provides an isolated DNA molecule comprising the test oligonucleotide as hereinbefore defined.

Viewed from a yet further aspect, the present invention provides any agents identified by the aforementioned screen, and their use in combating diseases associated with Smad/TGFβ gene activation.

As a yet further aspect, the present invention provides any agents which inhibit or activate transcriptional activity or binding of one or more Smad proteins with a promoter or enhancer implicated in the gene regulation of TGFβ or activin, said promoter comprising the nucleotide sequence 5′ WXYCAGACZ 3′ or a functional equivalent thereof, wherein in said nucleotide sequence W represents A or G, X represents G or T, Y represents C, A or G and Z represents A or C.

Such agents may be any type of molecule including small organic molecules, proteins or polypeptides, or nucleic acid molecules. Agents identified as having a desired effect may be tested further in appropriate models of fibrosis, wound healing, cancer, haematopoiesis, neuroprotection, immunity or inflammation.

### EXAMPLES

In a method known as transcriptional screening, the invention may be used to screen agents that activate or inhibit the TGFβ or activin transduction pathway from cell membrane to the nucleus that in fine leads to CAGA box-mediated transcriptional regulation.

A reporter vector can be generated by cloning a transcriptional region bearing CAGA boxes in a plasmid containing a reporter gene, for instance, the firefly luciferase, so that this transcriptional CAGA containing region controls the transcription of the reporter gene. In particular, the PAI-1 promoter can be cloned upstream of the firefly iuciferase gene.

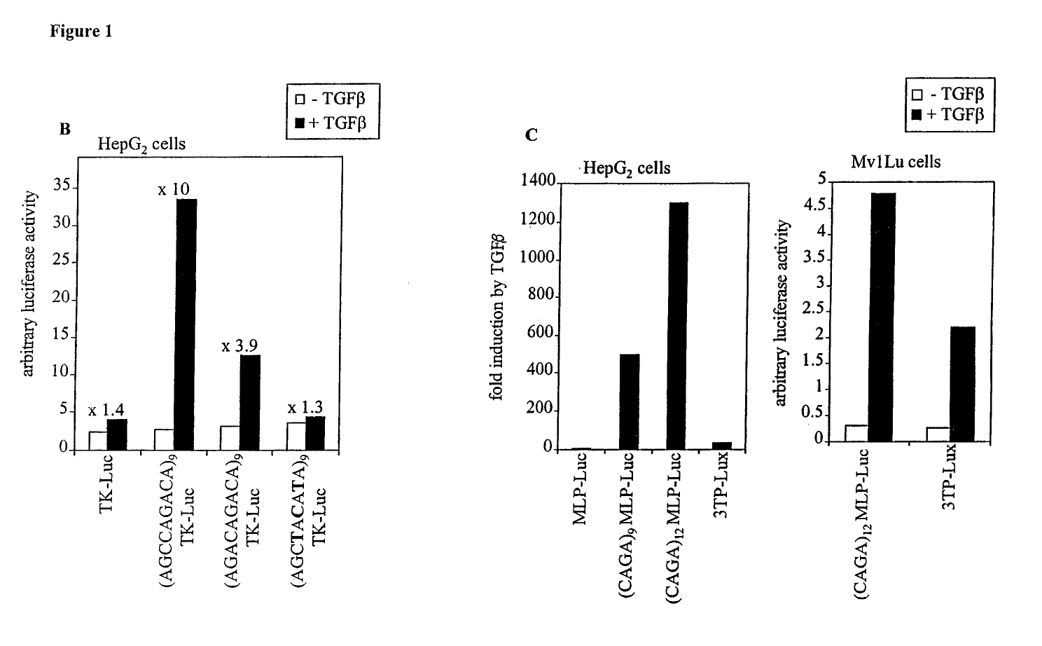

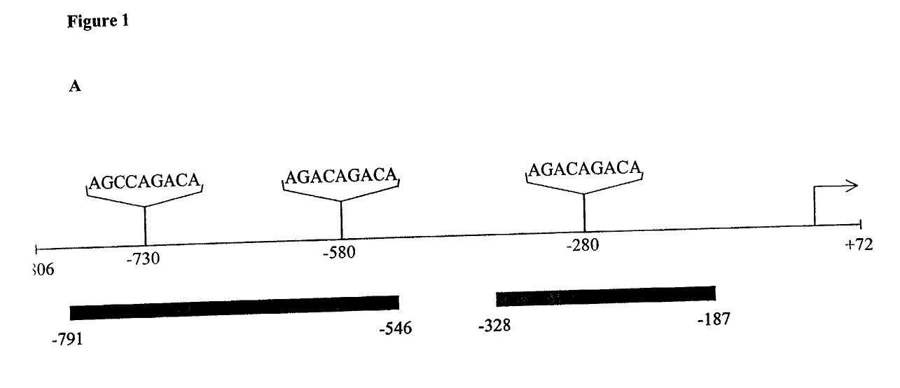

Alternatively, an artificial construct can be synthesized in which chemically generated oligonucleotides containing CAGA sequences are cloned in a promoter or an enhancer configuration so that they control the transcription of the firefly luciferase gene. Such constructs are described in FIG. 1 where CAGA oligonucleotides are cloned upstream of the TK or MLP promoters. This TGFβ-inducible CAGA sequence-containing reporter vector has to be transfected into eukaryotic cells, preferably into a mammalian cell line, for instance, the HepG2 cell line, by various and classical means such as calcium-phosphate precipitate, DEAE-dextran, liposome-mediated or electroporation methods.

Preferably, the transfection generates a clonal cell line that stably expresses the CAGA boxes containing reporter transgene. This may be obtained by co-transfection of a resistance plasmid encoding for a resistance gene to drugs such as neomycin or hygromycin, and selection for transfected cells that have acquired, by stable integration of the resistance plasmid, resistance to the mentioned drug.

Preferably, the stable cell-line has stably integrated another transgene, such as renilla luciferase for instance, whose expressed product possesses a measurable activity. The expression of this transgene should not be regulated by TGFβ or activin, i.e. it should not contain CAGA sequences in its regulatory regions. For instance, the renilla-luciferase gene can be transcribed from the RSV (Rous Sarcoma Virus) promoter or SV (Simian Virus 40) promoter. When screening for pharmacological agents that modify the expression of the firefly luciferase transgene, i.e. have an action through the CAGA sequences, the expression of the renilla luciferase transgene serves as a specificity control. This means that an agent acting specifically through CAGA boxes-mediated transcription will have an effect on the firefly luciferase activity but not on the renilla luciferase activity. In particular, when screening for inhibitors of CAGA boxes-mediated transcription, the renilla luciferase activity discriminates between agents that specifically inhibit CAGA boxes-mediated transcription from those that are toxic.

The assay mixture comprises transfected cells incubated in an adequate cell culture medium and one or several candidate pharmacological agents. In the case where inhibitors are screened, the cell culture medium contains TGFβ or activin (preferably at a concentration between 0.1 ng/mL to 50 ng/mL) in order to activate CAGA sequences-mediated transcription. The presence of TGFβ or activin is dispensable in the case where activators are screened. A difference in the firefly luciferase activity between a mixture where one or several candidate pharmacological agents are present and a mixture without such a candidate agent indicates that this or these agents are able to modulate the transcriptional activity mediated by the binding of Smad proteins on the CAGA sequence.

Candidate agents encompass numerous chemical classes, though typically they are organic compounds, preferably small organic compounds with a molecular weight often comprised between 50 and 2500, more preferably less than about 1000. Candidate agents are also found among biomolecules including peptides, saccharities, fatty acids, steroids, purines, pyrimidines, derivatives, structural analogs or combinations therof, and the like. Candidate agents are obtained from a wide variety of sources including random and directed synthesis, combinatorial chemistry and libraries of synthetic or natural compounds.

The method described herein is particularly suited to high-throughput screening. In order to automate the process, transfected cells are seeded and cultured in 96 wells or 384 wells microplates. A computer controlled electromechanical robot, comprising an axial rotable arm, is programmed to execute the different steps of the test: cells seeding, incubation with medium in the presence or the absence of TGFβ or activin, incubation with test pharmacological agents, cells washings and luciferases activities revelation. Luciferases activities are read with classical methods using commercially available kits, preferably with a dual injector luminometer connected to the robot and able to read microplates.

The invention will now be described with reference to the following non-limiting examples in which:

FIG. 1: The CAGA box is a TGFβ-inducible DNA element.

FIG. 1A: In the human PAI-1 promoter, two regions, depicted by heavy bars, have been described to respond to TGFβ. The sequences of the three CAGA boxes found in this promoter are given.

FIG. 1B: HepG2 cells were transfected with different vectors containing nine copies of the CAGA sequence cloned upstream of the HSV1-Thymidine Kinase promoter (TK). AGCCAGACA is the sequence found at position −730 in the PAI-1 promoter and AGACAGACA is the sequence of the two other CAGA boxes of the PAI-1 promoter (positions −580 and −280). The last construct contains mutated CAGA boxes on three pb as indicated. Luciferase activities are shown and fold inductions by TGFβ are indicated.

FIG. 1C: HepG2 and Mv1Lu cells were transfected with p3TP-Lux or a vector containing nine or twelve copies of the CAGA box upstream of the minimal Adenovirus Major Late Promoter (MLP). Fold inductions by TGFβ are given for HepG2 cells. Basal and TGFβ-induced luciferase levels are shown for Mv1Lu transfected cells.

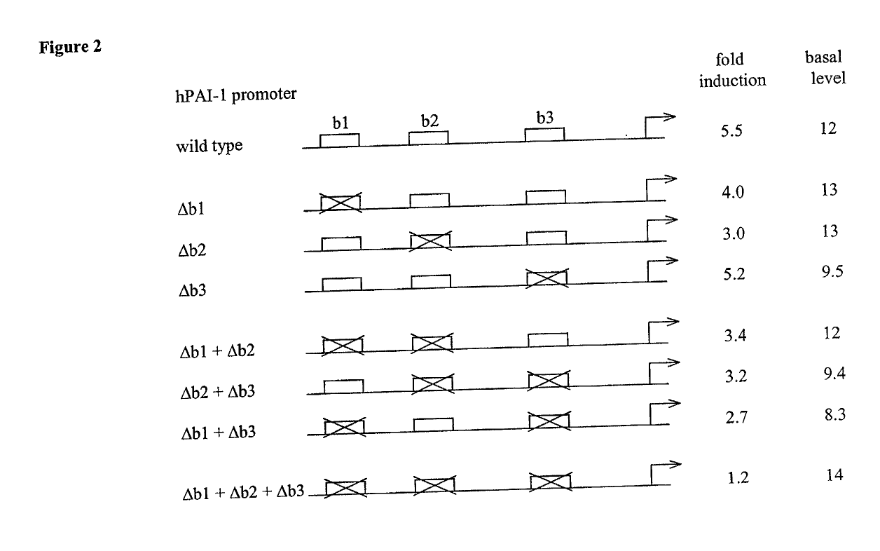

FIG. 2: The CAGA box of the human PAI-1 promoter is necessary for induction by TGFβ. Mutations of the CAGA boxes in the PAI-1 promoter were introduced by site-directed mutagenesis. The wild type AG(C/A)CAGACA sites were replaced by the mutated AG(C/A)TACATA sequence. The mutated boxes are represented by a crossed rectangle. Basal levels in the absence of TGFβ and fold inductions in the presence of TGFβ in transfected HepG2 cells are given.

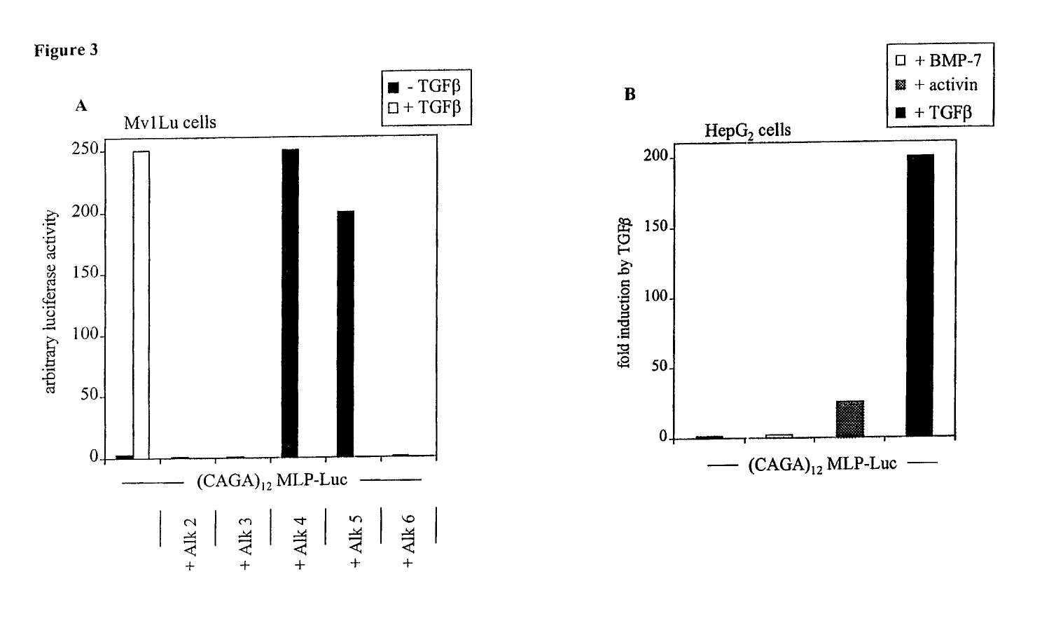

FIG. 3: The CAGA box responds to TGFβ and activin signalling but not to BMPs pathways.

FIG. 3A: Mv1Lu cells were cotransfected with a (CAGA)₁₂-MLP-Luc reporter construct and expression vectors encoding for constitutively activated versions of serine/threonine kinase receptors specific of TGFβ, activin or BMPs signalling. Alk-2 is the ActR-I receptor, Alk-3 the BMPR-1A receptor, Alk-4 the ActR-1B receptor, Alk-5 the TGFβR-1 receptor and Alk-6 the BMPR-1B receptor.

FIG. 3B: HepG2 cells were transfected with a (CAGA)₁₂-MLP-Luc reporter construct and induced by BMP-7, activin or TGFβ (respectively 100 ng/mL, 20 ng/mL and 10 ng/mL).

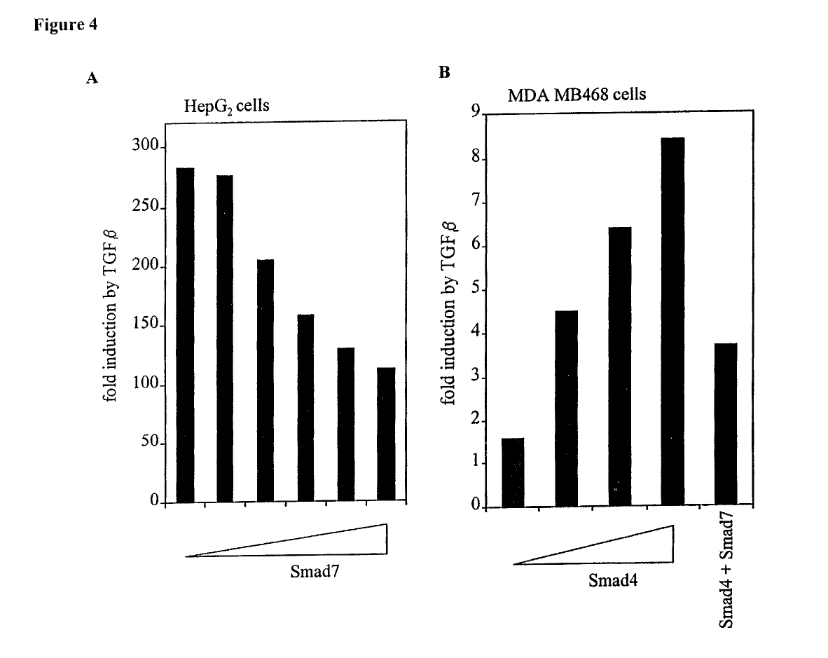

FIG. 4: Smad proteins are involved in TGFβ-induced transcription mediated by the CAGA box.

FIG. 4A: HepG2 cells were cotransfected with a (CAGA)₉-MLP-Luc reporter construct and increasing amounts (0, 10, 15, 20, 30 and 40 ng) of an expression vector encoding for the Smad7 inhibitory protein.

FIG. 4B: MDA-MB468 cells were transfected with a (CAGA)₉-MLP-Luc reporter construct and increasing amounts (0, 250, 500, 750 ng) of an expression vector encoding for the Smad4 protein. 250 ng of Smad7 expression vector with 500 ng of Smad4 expression construct were cotransfected when indicated.

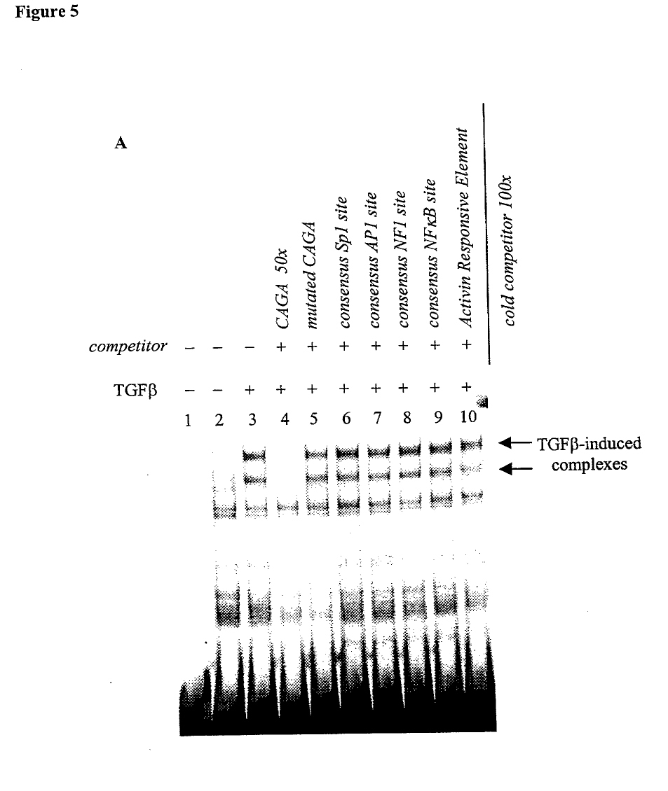

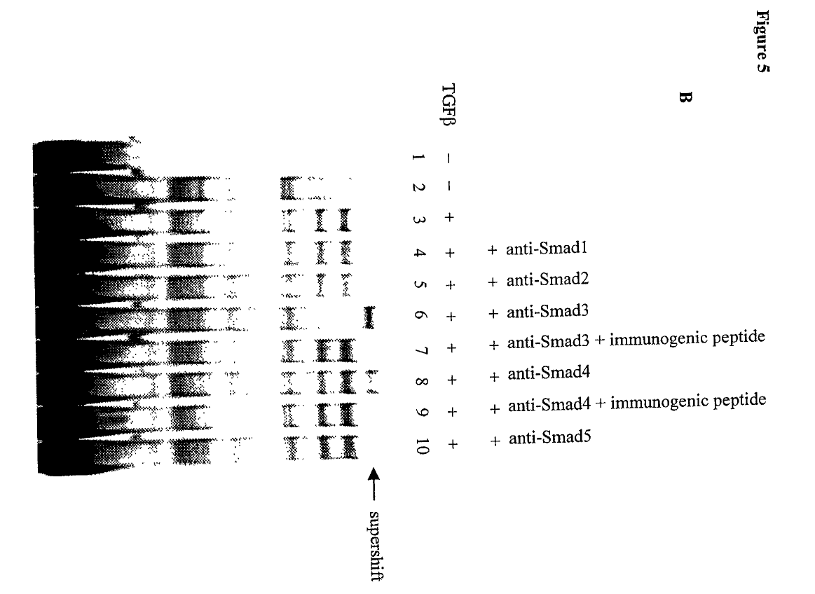

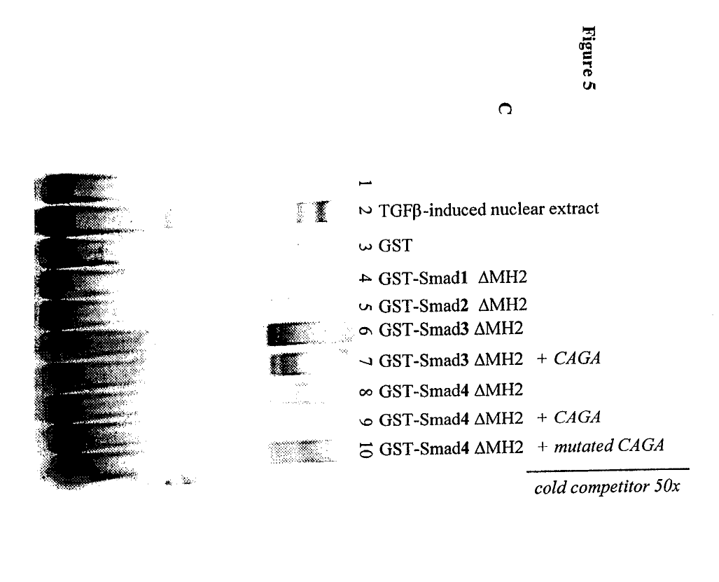

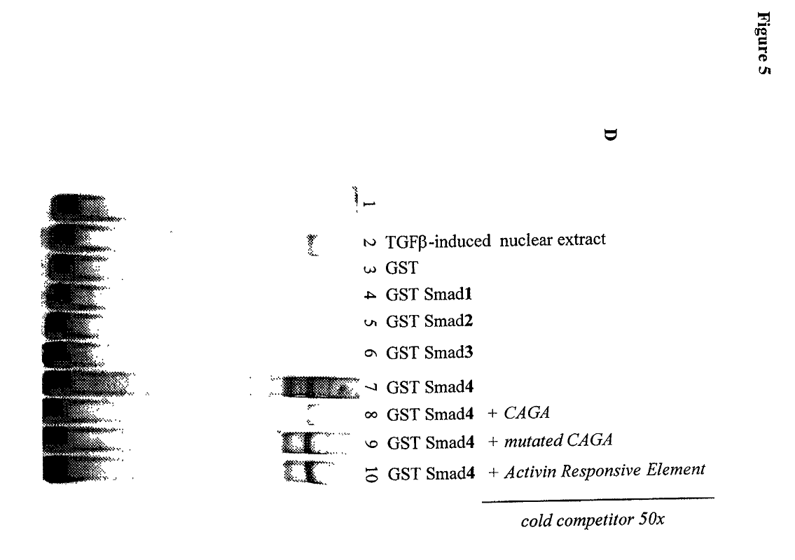

FIG. 5: Smad3 and Smad4 bind directly to the TGFβ-inducible CAGA box.

FIG. 5A: an EMSA was performed using a ³³P-labelled probe containing the CAGA sequence and nuclear extract from HepG2 cells induced 30 min by TGFβ or not induced. Bands corresponding to specific TGFβ-induced complexes are indicated. 50 or 100 molar excess of various cold oligonucleotides were added as competitors, including the wild type and mutated CAGA sequences.

FIG. 5B: Specific anti-Smad antisera were incubated with TGFβ-induced HepG2 nuclear extracts before mixing with the CAGA probe. The supershifted complexes are indicated. The antigenic peptides used to generate the reactive anti-sera were added in lane 7 and 9 to show the specificity of the anti-Smad3 and anti-Smad4 antisera.

FIG. 5C: E. coli expressed GST-Smad1, 2, 3 and 4 proteins, deleted of the conserved carboxy-terminal MH2 region, were incubated with a ³³P-labelled CAGA probe. 50 molar excess of cold oligonucleotide competitors were added when indicated. Nuclear extracts of TGFβ-treated HepG2 cells have been added to the probe in lane 2 to locate the nuclear DNA-binding complex.

FIG. 5D depicts a similar experiment where full length Smad proteins, fused to the GST domain, produced in bacteria were used.

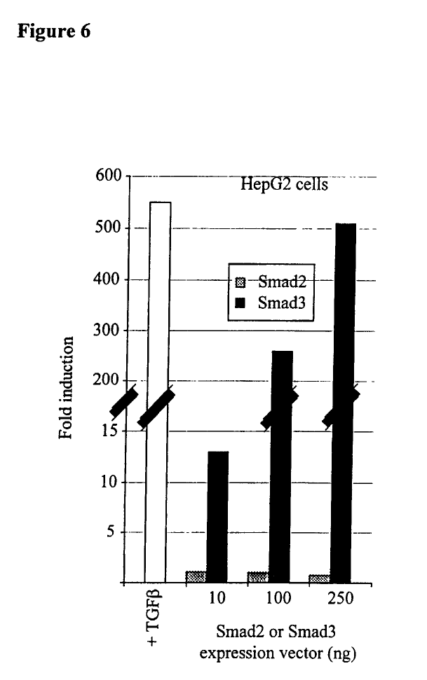

FIG. 6: Smad3 overexpression mimics TGFβ activation of reporter vectors whereas Smad2 overexpression does not. HepG2 cells were transiently transfected with the (CAGA)₉ MLP-Luc reporter vector. Cells co-transfected with Smad expression vectors, as indicated, were serum-starved but not treated with TGFβ.

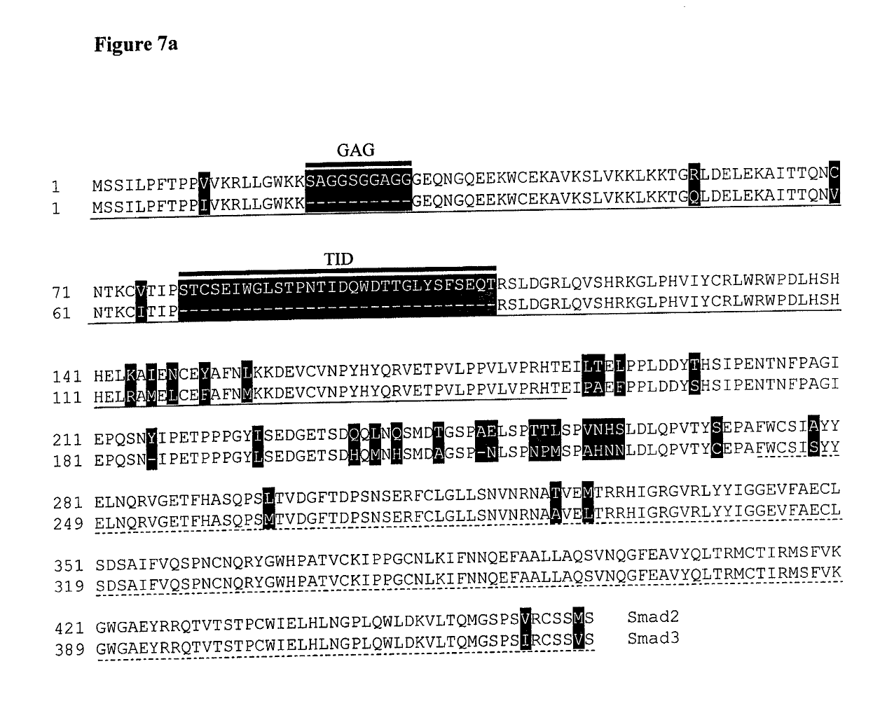

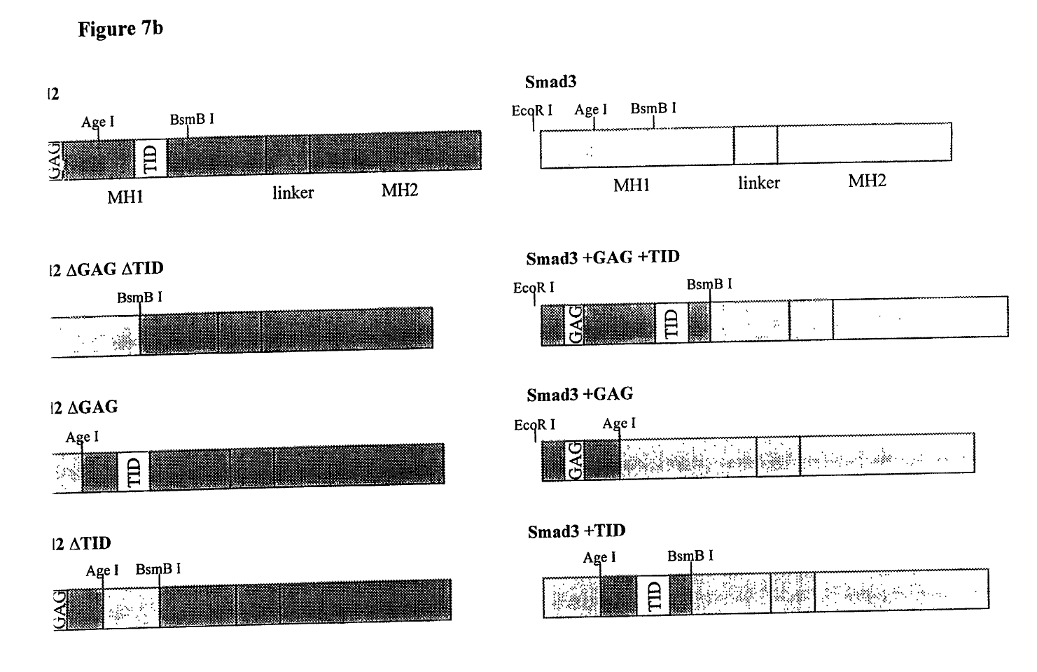

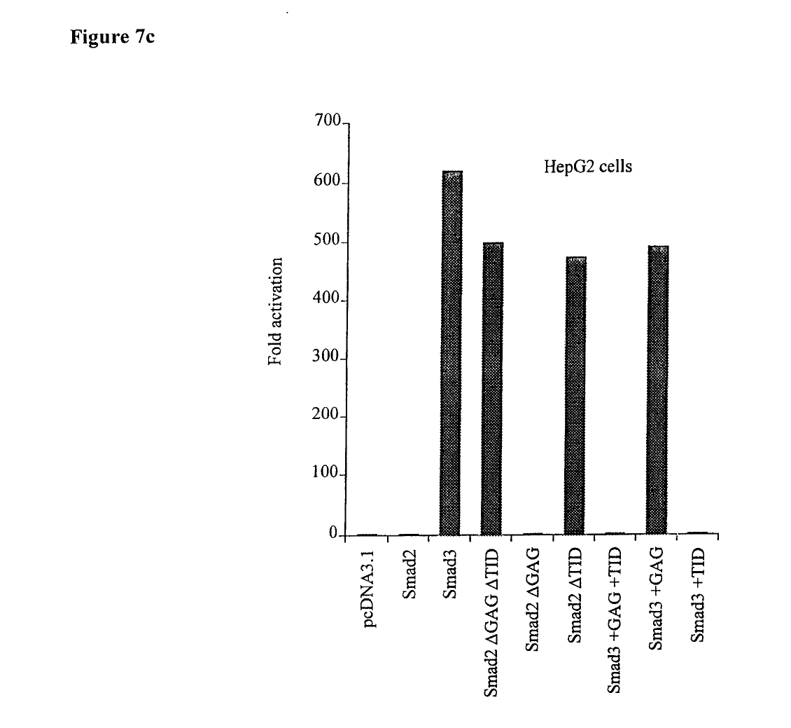

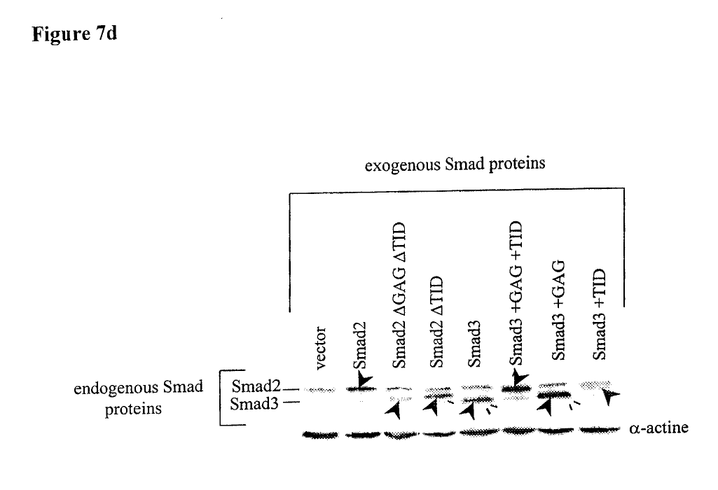

FIG. 7: Mapping of the Smad2 domain responsible for transcriptional inactivity.

FIG. 7A: Human protein sequences of Smad2 and Smad3. Black boxes encompass differences between the sequences of the two proteins. MH1 and MH2 domains are underlined respectively with a straight and a dotted line. The GAG and the TID domains are also indicated.

FIG. 7B: Schematic of Smad2 and Smad3 domain swap chimeras.

FIG. 7C: Induction of (CAGA)₉ MLP-Luc reporter vector by Smad2 and Smad3 mutants in HepG2 cells. Cells were transfected with the (CAGA)₉ MLP reporter vector along with equal concentrations of the indicated mutant constructs and assayed for luciferase activities in the absence of TGFβ.

FIG. 7D: Western blot analysis of HepG2 cellular extracts expressing Smad2 or Smad3 mutants. After transfection, cells were lysed with the lysis buffer provided with the Dual-Luciferase Assay Kit (Promega), proteins were separated on 8.5% SDS-PAGE then blotted with an anti-Smad2/Smad3 polyclonal antibody (sc-6032, Santa Cruz). Lysates were also immunoblotted with an anti-β-actine polyclonal antibody (sc-1615, Santa Cruz) to assess equal protein loading. The primary antibodies were revealed by chemoluminescence with a secondary antibody coupled to horse peroxidase.

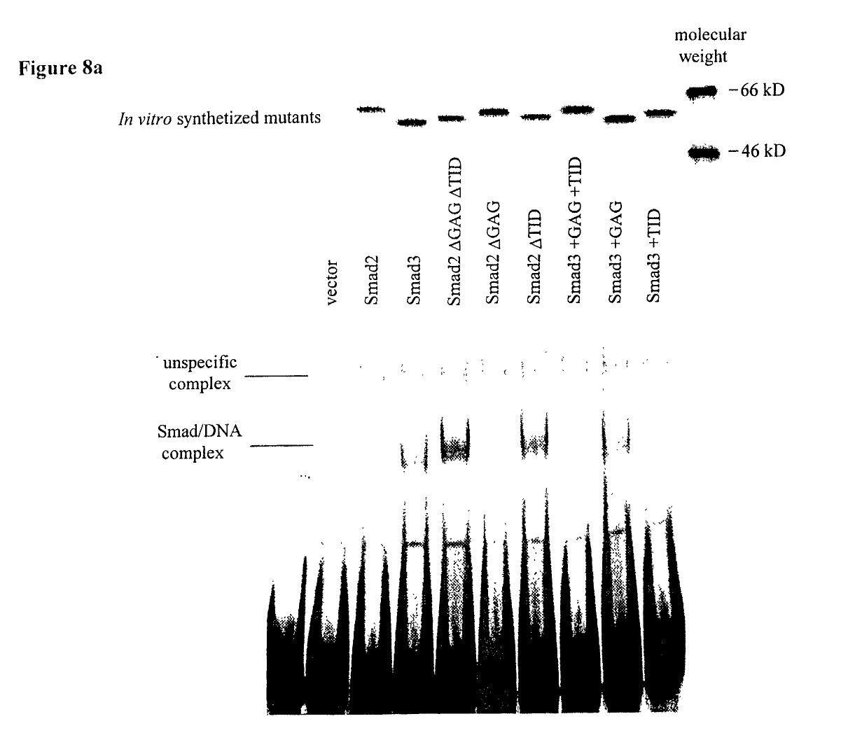

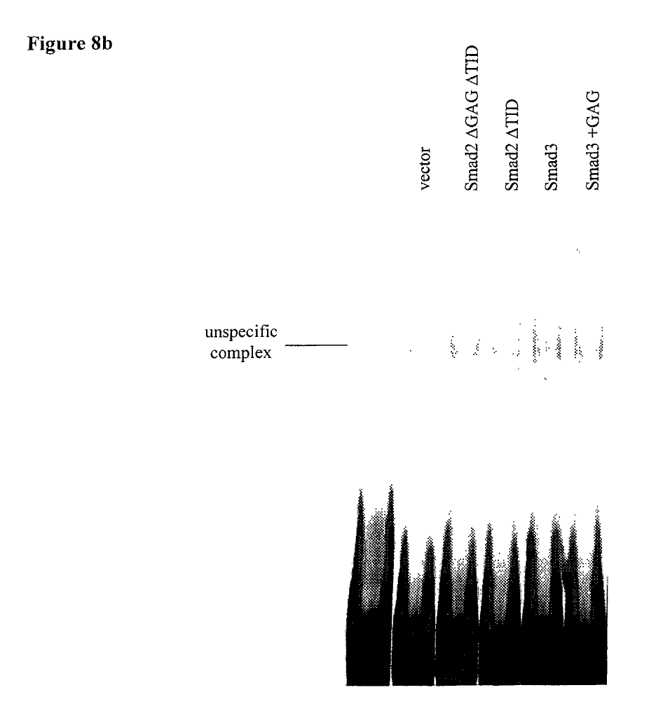

FIG. 8: The TID domain prevents Smad2 from binding to the CAGA sequence.

FIG. 8A: SDS-PAGE analysis of Smad2 and Smad3 mutants translated in vitro (upper panel) and gel shift assays using these in vitro translated proteins on a CAGA oligonucleotide (lower panel).

FIG. 8B: Gel shift assay using Smad mutants on a mutated CAGA probe.

### Experimental Methods

Plasmids Constructs

CAGA reporter vectors were generated using pGL3 basic plasmid (Promega). TK or MLP promoters were PCR-amplified and inserted between the BgI II and Hind III sites. The CAGA boxes-containing oligonucleotides were cloned into the Xho I site. The sequences of the oligonucleotides cloned are:

CAGA boxes containing oligonucleotides:

CAGA Boxes Containing Oligonucleotides:
5′ TCGAGAGCCAGACAAAAAGCCAGACATTTAGCCAGACAC 3′
3′ CTCGGTCTGTTTTTCGGTCTGTAAATCGGTCTGTGAGCT 5′
5′ TCGAGAGACAGACAAAAAGACAGACATTTAGACAGACAC 3′
3′ CTCTGTCTGTTTTTCTGTCTGTAAATCTGTCTGTGAGCT 5′
CAGA Mutant Oligonucleotide:
5′ TCGAGAGCTACATAAAAAGCTACATATTTAGCTACATAC 3′
3′ CTCGATGTATTTTTCGATGTATAAATCGATGTATGAGCT 5′

The PAI-1—Luc vector was generated by insertion of the PCR-amplified—806+72 fragment of the human PAI-1 promoter in the Sac I/BgIII sites of the pGL3-Basic vector (Promega). The site-directed mutagenesis in the human PAI-1 promoter was performed using the QuickChange Site-Directed Mutagenesis Kit (Stratagene) according to the manufacturer protocol. In order to generate Smad2 and Smad3 mutants containing or not GAG and TID domains, Age I restriction site was inserted by site-directed mutagenesis (QuickChange Site-Directed mutagenesis kit, Stratagene) in the expression vectors encoding Smad2 and Smad3. BsmB I restriction sitewas inserted similarly in Smad3 expression vector. Insertion of restriction sites did not modify the amino-acid sequence of the proteins. All the constructs were sequence-checked.

Cell Culture

The human hepatoma cell line HepG2 (HB 8065), the human breast adenocarcinoma cell line MDA-MB468 (HTB 132) and the Mv1Lu mink lung epithelial cell line (CCL 64) were purchased from the American Type Culture Collection. HepG2 and Mv1Lu cells were grown in a 5% CO₂-95% air atmosphere in BME or MEM medium respectively (Life Technologies, Inc.) supplemented with 10% fetal bovine serum, 10 mM sodium pyruvate, 100 IU/mL penicillin, 100 μg/mL streptomycin and 2 mM L-glutamine (complete medium). MDA-MB468 cells were grown in a 7.5% CO₂-92.5 % air atmosphere in DMEM/F12 (1:1) medium (Life Technomogies, Inc.) with 10% fetal bovine serum, 100 IU/mL penicillin, 100 μg/mL streptomycin and 2 mM L-glutamine (complete medium).

Transfection and luciferase Assays

HepG2 and MDA-MB468 cells were transiently transfected, with the indicated constructs and the internal control pRL-TK vector, using the calcium phosphate co-precipitation method. When increasing amounts of expression vectors were transfected, total DNA was kept constant by addition of pCMV5. Cells were serum starved for 8 h before stimulation with 7 ng/mL of human recombinant TGFβ1 (R&D) and luciferases activities were quantified 14 h later using the Dual Luciferase Assay (Promega). For activin and BMP-7 (Creative Biomolecules) induction, respectively 20 ng/mL and 100 ng/mL were used. Values were normalized with the renilla luciferase activity expressed from pRL-TK. Mv1Lu cells were transfected using the DEAE-dextran method. Luciferase values shown in figures are representative of transfection experiments done at least three times.

Nuclear Extracts

Nuclear extracts were prepared from control and TGFβ-treated HepG2 cells. Cells were harvested thirty minutes after treatment and processed according to Sadowski and Gilman's protocol (Sadowski and Gilman, 1993). Briefly, confluent cells from eight 100-mm dishes were washed with phosphate-buffered saline and scraped. After another washing, cells were suspended in 2 mL of cold buffer A (20 mM HEPES pH 7.9, 20 mM NaF, 1 mM Na₃VO₄, 1 mM Na₄P₂O₇, 0.13 μM okadaic acid, 1 mM EDTA, 1 mM EGTA, 0.4 mM ammonium molybdate, 1 mM DTT, 0.5 mM PMSF and 1 μg/mL each leupeptin, aprotinin and pepstatin). The cells were allowed to swell on ice for 15 min then were lysed by 30 strokes of Dounce all glass homogenizer. Nuclei were pelleted by centrifugation and resuspended in 600 μL of cold buffer C (buffer A, 420 mM NaCl and 20% glycerol). The nucleus membrane was lysed by 15 strokes of Dounce all glass homogenizer. The resulting suspension was stirred for 30 minutes at 4° C. The clear supernatent was aliquoted and frozen at −80° C.

Electrophoretic Mobility Shift Assays

Oligonucleotides were end-labeled with [α-³³P]dCTP and [α-³³P]dATP using the Klenow fragment of DNA polymerase. Binding reactions containing 10 μg of nuclear extracts or 400 ng of GST-Smad proteins or 16 μL of in vitro translated Smad proteins and 2 ng of labeled oligonucleotides were performed for 20 min at 37° C. in 18 μL of binding buffer (20 mM HEPES pH 7.9, 30 mM KCl, 4 mM MgCl₂, 0.1 mM EDTA, 0.8 mM NaPi, 20% glycerol, 4 mM spermidine, 3 μg poly dl-dC). Protein-DNA complexes were resolved in 5% polyacrylamide gels containing 0.5× TBE. The sequence of the double stranded oligonucleotide used as a probe was:

| 5′ TCGAGAGCCAGACAAGGAGCCAGACAAGGAGCCAGACAC | |
|---|---|
| CTCGGTCTGTTCCTCGGTCTGTTCCTCGGTCTGTGAGCTC | 5′ |

The sequence of the competitor CAGA mutant oligonucleotide was:

5′ TCGAGAGCTACATAAAAAGCTACATATTTAGCTACATAC 3′
3 CTCGATGTATTTTTCGATGTATAAATCGATGTATGAGCT 5′

Competitor oligonucleotides containing other transcription binding sites are:

| | |
|---|---|
| 5′ TCGAGGCTGCCCTAAAATGTGTATTCCATGGAAATGTCTGCCCTTCTCTC | 3′ |
| 3′ CCGACGGGATTTTACACATAAGGTACCTTTACAGACGGGAAGAGAGAGCT | 5′ |

AP-1 site
5′ CCGGGATGACTCAGC 3′
3′ CTACTGAGTCGGGCC 5′
NF-1 site
5′ CCGGTTTGGATTGAAGCCAATATG 3′
3′ AAACCTAACTTCGGTTATACGGCC 5′
Sp1 site
5′ TCGAGGACAGGGGGCGGAGCCTC 3′
3′ CCTGTCCCCCGCCTCGGAGAGCT 5′

In gel shift experiments realized with in vitro translated proteins, Smad proteins were produced using the TNT T7 Quick Coupled Transcription/Translation System (Promega) according to the manufacturer's protocol. The in vitro synthesized proteins labeled with [³⁵S]methionine were controlled by SDS-PAGE and autoradiography before utilisation in EMSA.

Production and Purification of Smad Fusion Proteins

The full-length Smad proteins and the MH2-deletion mutants fused to GST were expressed in E coli and partially purified by column chromatography using Pharmacia's protocol. Briefly, bacteria were grown in 2× YTA medium and induced with 0.1 mM IPTG. After sonication, the GST-fusions were isolated using Glutathione Sepharose 4B, washed three times, eluted, then dialysed against PBS supplemented with 2 mM DTT and 0.5 mM PMSF.

### Experimental Results

The CAGA Box Is a TGFβ-Inducible DNA Element

We raised the possibility that a common sequence motif could be present in the TGFb-responsive regions that have been identified along the human PAI-1 promoter. To address this question, we looked for a short DNA homology element and noticed that the sequence AG(C/A)CAGACA was found in three copies at positions −730, −580 and −280 in the human PAI-1 promoter in regions that have been shown to mediate TGFβ-transcriptional induction (FIG. 1A). We named this sequence the CAGA box and cloned it in a transcriptional reporter system to determine its involvement in TGFβ-induced transcription. When cloned in multiple copies upstream of the thymidine kinase (TK) promoter, this DNA sequence confers TGFβ-mediated induction in HepG2 cells (FIG. 1B), without affecting the basal activity of the vector. Similar results were observed in Mv1Lu cells (FIG. 1C) or in NIH3T3 cells (data not shown). Several hundred TGFβ-mediated fold induction in HepG2 cells were obtained when multiple CAGA boxes were cloned upstream of a minimal promoter consisting of the TATA box and the initiator sequence of the adenovirus major late promoter (MLP) (FIG. 1C). This induction was lower with the widely used TGFβ-responsive p3TP-Lux plasmid. It is noteworthy that p3TP-Lux contains the −740/−636 region of the PAI-1 promoter bearing the −730 CAGA box. As a control of specificity, the mutated sequence AGCTACATA, containing three point mutations relative to the original sequence, was unable to confer TGFb induction to the TK promoter (Figure 1C).

Mutation of the CAGA Boxes in the Human PAI-1 Promoter Abolishes TGFβ Responsiveness

The wild type human PAI-1 promoter contains three CAGA boxes. To explore the biological significance of these boxes in the TGFβ-mediated induction of this promoter, we mutated each of the three native sequences by introducing the TGFβ-non-induced mutant sequence (FIG. 2). Mutation of one of the three sites led up to 45% decrease of TGFβ induction compared to the wild type promoter (FIG. 2 see Δb1, Δb2 and Δb3 mutants). With two sites, the decrease was higher (FIG. 2 see Db1+Db2, Δb1+Δb3 and Δb2+Δb3 mutants) and when all three sites were mutated, the PAI-1 promoter was almost unable to respond to TGFβ (FIG. 2, see Δb1+Δb2+Δb3 mutant). These CAGA boxes appear not to significantly control the basal activity of the promoter since, in the absence of TGFβ, the rate of transcription of the mutant promoters and the wild-type PAI-1 promoter were comparable.

The CAGA Box Responds to TGFβ and Activin, but Not to BMP

Specific serine/threonine kinase type I receptors transduce intracellular signalling of TGFβ family members; BMPR-IA (ALK-3), BMPR-IB (ALK-6) and ActR-I (ALK-2) are BMP type I receptors, whereas TGFβ and activin signal through TβR-1 (ALK-5) and ActR-IB (ALK4), respectively. To test the specificity of the CAGA box relative to TGFβ superfamily members, we transfected Mv1Lu cells, which are responsive to TGFβ, activin and BMP-7, with expression vectors encoding for constitutively activated versions of the type I receptors. As shown in FIG. 3A, expression of ALK-4/T206D and ALK-5/T204D led to transcriptional activation of the CAGA box reporter vector. In contrast, expression of ALK-2/Q207D, ALK-3/Q233D and ALK-6/Q204D did not show any effect, demonstrating that the CAGA sequence is activated by TGFβ and activin, but not by BMP-induced signalling in Mv1Lu cells. Similar results were obtained in HepG2 cells with transfection of constitutively activated versions of type I receptors (data not shown). In order to test more physiological conditions, we transfected HepG2 cells, which are responsive to activin and BMP-7, with a CAGA box reporter vector and incubated the cells with activin and BMP-7 (OP-1). As shown in FIG. 3B, the CAGA boxes containing reporter was induced respectively 25 and 200 fold in the presence of activin and TGFβ whereas BMP-7 did not show any significant effect (2 fold induction). Thus, CAGA boxes respond specifically to activin and TGFβ but not to BMP signalling.

Smad Proteins Participate in TGFβ-Induced Transcription Mediated by the CAGA Box

To examine whether Smad proteins were involved in the TGFβ-induced transcriptional activation observed with the CAGA box, we cotransfected HepG2 cells with a CAGA reporter construct and an expression vector encoding for the Smad7 protein, known to inhibit TGFβ/Smad-mediated transcriptional effects. As shown in FIG. 4A, overexpression of Smad7 leads to a 50% inhibition of TGFβ-induced transcription of the CAGA box reporter construct. MDA-MB468 cells, derived from a breast cancer, are human epithelial cells deficient for endogenous Smad 4 expression. In these cells, TGFβ has no effect on a CAGA reporter construct (FIG. 4B). However, cotransfection of an expression vector encoding for Smad4 restores TGFβ transcriptional induction of the CAGA boxes containing vector, demonstrating that Smad4 is necessary for the TGFβ transcriptional effect mediated by this sequence.

Smad3 and Smad4 are Present in the Transcription Factor Nuclear Complexes that Bind to the CAGA Box

In a next step, we performed electrophoretic mobility-shift assays (EMSA) using HepG2 nuclear extracts in an attempt to characterize the DNA-binding activity on the TGFβ-responsive CAGA sequence. We could identify binding complexes present only with nuclear extracts from cells induced by TGFβ (FIG. 5A, compare lanes 2 and 3). Maximum binding requires a TGFβ-induction time of 30 min but the complex can be clearly observed after a 10 min induction (data not shown). This suggests that a de novo protein synthesis is not necessary and that an already existing factor is rapidly and post-translationally modified or translocated into the nucleus. This DNA-binding complex is specific since an excess of the cold CAGA oligonucleotide, but not of the mutated box, displaces the corresponding band (FIG. 5A, lines 4 and 5). Furthermore, this complex does not contain transcription factors proposed as potential mediators of TGFβ/activin signalling such as Sp1, AP-1, NF-1 or FAST-1 since it is not displaced by the corresponding DNA sequences to which these transcription factors bind (FIG. 5A, lanes 6 to 10). To examine whether Smad proteins were present in the CAGA binding complex, nuclear extracts were incubated with specific antisera to Smad1 through Smad5. We could detect a supershift of the TGFβ-dependent binding complex with anti-Smad3 and anti-Smad4 antisera (FIG. 5B, lanes 6 and 8). These supershifts were competed by addition of the immunogenic peptides that was used to generate the antisera, proving the specificity of the antibody recognition (FIG. 5B, lanes 7 and 9). Since addition of anti-Smad1, anti-Smad2 and anti-Smad5 antisera have no effect (FIG. 5B, lines 4, 5 and 10), we conclude that the CAGA box DNA-binding nuclear complex contains the TGFβ/activin signalling Smad 3 and Smad4 proteins, but not Smad protein nor the BMP signalling Smad1 and Smad5 proteins. This is in agreement with the transfection experiments showing that the CAGA reporter construct is activated by the TGFβ and activin receptors which activate Smad3, but not by the BMP receptors which signal through Smad1 and Smad5 (see FIGS. 3A and 3B).

Smad3 and Smad4 Bind Directly to the TGFβ-Inducible CAGA Box

The previous gel shift experiments that we have described demonstrate the presence of Smad3 and Smad4 in the nuclear CAGA sequence-binding complex, but cannot determine whether binding of Smad3 and Smad4 to DNA is direct or not. To address this issue, we used E. coli expressed GST-Smad fusion proteins in EMSA. As shown in FIG. 5C, Smad3 and Smad4 deleted of the MH2 domain, bound directly and specifically to a CAGA box containing probe. In line with the supershift experiments, the Smad1 ΔMH2 and Smad2 ΔMH2 proteins failed to bind DNA. Furthermore, and in opposition with the example of the Drosophila Mad protein, the full length Smad4 protein produced in bacteria did possess a direct and specific DNA-binding activity on the CAGA sequence (FIG. 5D), whereas full length GST-Smad1, GST-Smad2 and GST-Smad3 are unable to bind DNA.

Smad2 does not Activate GAGA-Mediated Transcription

As shown in FIG. 6, TGFβ activation on a CAGA reporter can be mimicked by transfection of an expression vector of Smad3 in HepG2 cells. However, transfection of the Smad2 protein, which shares an overall 92% identity with Smad3, had no effect on the CAGA-mediated transcription, indicating that Smad2 and Smad3 are not functionally equivalent. MH1 domain of Smad3 is sufficient for specific DNA-binding to the CAGA sequence (see FIG. 5C). A comparison between Smad2 and Smad3 MH1 domain reveals that the main difference is the presence of two stretches of amino acids in Smad2 that are lacking in Smad3 (FIG. 7A). We termed GAG the short N-′ terminal amino-acid sequence containing 10 residus (essentially glycine and serine) comprised between Ser²¹ and Gly³⁰. The larger sequence, long of thirty-residus from amino acid Ser⁷⁹ to Thr¹⁰⁸ and rich in serine and threonine was called TID. In order to determine whether these sequences are implicated in the lack of transcriptional activity of Smad2, we generated a Smad2 protein deleted in both sequences (FIG. 7B). This mutant transfected in HepG2 cells activated the CAGA reporter to a comparable level than wild type Smad3 (FIG. 7C). This Smad2 ΔGAG ΔTID mutant shows that domains GAG or TID are involved in the functional difference observed between Smad2 and Smad3. In a next step, we tried to determine if this transcriptional difference could be attributed to a single domain. To address this question, we deleted GAG (Smad2 ΔGAG) or TID (Smad2 ΔTID) sequences in Smad2 and tested the effect of mutants on CAGA reporter vector. As shown in FIG. 7C, Smad2 ΔTID mutant was clearly able to activate the CAGA reporter, indicating that the TID domain was involved in the absence of transcriptional ability of Smad2. We could not observe any activation of the CAGA reporter with Smad2 ΔGAG. However, we could not conclude from this experiment that the GAG domain is not involved in this absence of transcriptional activation since we were unable to detect expression of this mutant by western blot (FIG. 7D and data not shown).

In order to complement the results obtained with Smad2 deletion mutant, we introduced the GAG or TID domains in Smad3. In line with the previous data, the Smad3 mutants containing the TID sequence (i.e. Smad3 +GAG +TID and Smad3 +TID) were unable to activate the CAGA reporter, showing again the implication of this sequence. It is noteworthy that these transcriptionaly inactive mutants were expressed in the cells since they were detected in western blot assays (FIG. 7D). Introduction of the single GAG domain into Smad3 did not modify its transcriptional capacity (see Smad3 +GAG, FIG. 7C). These results clearly indicate that the transcriptional difference observed between Smad3 and Smad2 is due to the single TID domain and not to the GAG sequence.

TID Domain, Corresponding to Exon 3, Prevents Smad2 from Binding to the CAGA Sequence

The difference between Smad3 and Smad2 ability to activate transcription may be explained by different DNA-binding capacity. Indeed, since the TID domain is responsible for transcriptional difference between Smad2 and Smad3, it is possible that this domain prevents Smad2 from binding to DNA. In order to verify this hypothesis, we produced the Smad mutant proteins using an in vitro transcription/translation system and tested their DNA-binding capacities in gel shift assays. As shown in FIG. 8A, the full length wild-type Smad3, unlike Smad2, bound to the CAGA oligonucleotides. It is noteworthy that, in this experiment, Smad3 was not fused to the GST domain showing thus that somehow the GST domain modifies the DNA-binding ability of Smad3 (see FIG. 5D). This binding was specific since Smad3 was not able to bind to an oligonucleotidic probe containing a version of the CAGA sequence mutated in 3 nucleotides (FIG. 8B). In agreement with the transfection experiments, Smad2 deleted in both sequences (Smad2 ΔGAG ΔTID) and Smad2 ΔTID were able to bind to the CAGA probe whereas Smad2 ΔGAG did not. In total correlation with transcriptional activities observed previously, Smad3 +GAG bound CAGA oligonucleotides but introduction of TID domain into Smad3 (i.e Smad3 +TID and Smad3 +GAG+TID) hindered Smad3 from binding to DNA. Thus, the TID sequence prevents Smad2 from activating transcription by impeding its DNA-binding to the CAGA box.

Remarkably, the TID sequence present in Smad2 corresponds exactly to exon 3 (Takenoshita at al. Genomics, 1998, 48, 1-11). Furthermore, a version of Smad2 spliced in exon3 has been detected in human placenta (Takenoshita at al. Genomics, 1998, 48, 1-11). Possibly, this splicing event may be regulated and specific of certain cell types and conditions. Since this shorter form, unlike the full length Smad2, does not contain the TID domain, it activates transcription similarly than Smad3 and is redundant at least to some extent with Smad3, i.e. in its ability to bind and activate transcription from CAGA sequences.

Specific Example of CAGA-Mediated Transcriptional Screens

CAGA-Reporter Cellular Clones

Two cell lines containing stably integrated TGFβ-responsive CAGA box-containing reporters have been generated to perform high-throughput transcriptional screens. The first clonal cell line, clone F89, has been obtained by stable co-transfection in HepG2 cells of the (CAGA)₉MLP-Luc vector (firefly luciferase under the control of nine CAGA boxes cloned upstream of the minimal MLP promoter; described in FIG. 1) and the pRc/Renilla vector. pRc/Renilla vector contains the neomycinigeneticin gene resistance under the control of the SV40 promoter and the renilla luciferase gene driven by the RSV LTR. pRc/Renilla was obtained by cloning the HindIII/XbaI fragment of pRL-SV40 (Promega) containing the luciferase renilla gene into the HindIII/XbaI sites of the pRc/RSV vector (Invitrogen). The second clonal cell line, clone 1613, has been obtained by stable co- transfection in HepG2 cells of the wild-type human PAI-1-Luc reporter vector (firefly luciferase under the control of the human PAI-1 promoter; described in FIG. 2) and the pRc/Renilla plasmid. In both cases, HepG2 cells were stably transfected using the calcium phosphate co-precipitation method. Transfected cells were grown in the presence of 1 mg/mL geneticin (Gibco) in order to isolate geneticin resistant clones. F89 and 1613 clones were then isolated and amplified in the presence of 0.5 mg/mL geneticin to obtain sufficient amounts of cells for running high throughput screens.

Due to the presence of CAGA boxes in the transcriptional regulation region (i.e. promoter) controlling the expression of the firefly luciferase transgenes, both clones present an highly activated firefly luciferase activity in the presence of TGFβ in a dose-dependant manner. The activity of the renilla luciferase is almost not modified in the presence of TGFβ. Thus, the renilla luciferase activity can be used as an internal toxicity control.

Table 2 shows relative firefly luciferase activities (fold induction) observed in clones F89 and 1613 in the absence or presence of increasing amounts of TGFβ (value 1 corresponds to the relative firefly luciferase

TABLE 2

| TGFβ (ng/mL) | 0 | 0.2 | 0.5 | 1 | 5 | 10 |
|---|---|---|---|---|---|---|
| Clone F89 | 1 | 6 | 31 | 109 | 461 | 737 |
| Clone 1613 | 1 | 8 | nd | 26.2 | 43.5 | 50.1 |

Table 3 shows relative renilla luciferase activities (fold induction) observed in clones F89 and 1613 in the absence or presence of increasing amounts of TGFβ (value 1 corresponds to the relative renilla luciferase activity obtained in the absence of TGFβ):

TABLE 3

| TGFβ (ng/mL) | 0 | 0.2 | 0.5 | 1 | 5 | 10 |
|---|---|---|---|---|---|---|
| Clone F89 | 1 | 1.1 | 1.1 | 1.2 | 1.5 | 1.8 |
| Clone 1613 | 1 | 0.8 | nd | 1 | 1 | 1.3 |

Automated Robotic High Throughput Transcriptional Screen

The cellular assay has been automated in order to perform high-throughput screening in a 96 well-microplate format. The overall process is managed by a computer system (CLARA, Scitec) able to run actions in parallell and which controls peripheric equipments (i.e. axial rotable arm, carousel, cell-washer, pippetage station, cell incubator, luminometer. . .) and optimizes the temporal progression of the program. The general schedule used for this high-throughput screening is the following:

| | |
|---|---|
| 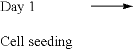 | 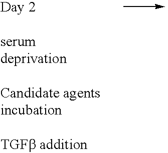 |

| | |
|---|---|
| 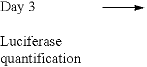 | 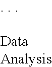 |

At day 1, 40 (96 well-) microplates are seeded, using a multidrop apparatus, with CAGA-reporter cells (i.e F89 or 1613) at a concentration of 35000 cells per well in 200 μl of serum-containing medium. These plates are placed in a cell incubator incorporated in the robotic line. This incubator is designed with a door that allows the entry of the axial rotable arm to handle the cell microplates.

8 to 24 hours later (Day 2), microplates containing the chemical compounds to be tested, diluted in 100% DMSO, are placed in a carousel and the cell-incubation procedure is launched. The computer system coordinates then the actions of different peripheric equipments in order to incubate the cells in the presence of TGFβ with the coumpounds to be tested.

Cells and compounds microplates are moved through the robotic line to the adequate peripheries by the axial rotatable arm. Cells are washed and incubated in a serum-free medium. The pippetage station realizes different operations including preliminary dilutions in order to incubate the cells with TGFβ and the chemical compounds to be tested. The final concentration of TGFβ (rhTGFβ-1 from R & D) used in the test is 1 ng/mL and the compounds are tested at a final concentration of 10 μl in a final concentration of DMSO of 1%. Cells are incubated with the compounds to be tested 15-30 mn prior the addition of TGFβ. The final volume of the test reaction is 150 μl. Wells A1 through H10 are the test wells and contain cells incubated with the chemical agents to be tested in the presence of TGFβ. Each well contains only one singular chemical compound and allows to test its effect on CAGA-mediated transcription. Columns 11 and 12 are kept for controls. Column 11 contains 8 wells where cells are incubated in the presence of TGFβ without chemical compounds. Column 11 determines of the reference TGFβ-induced firefly luciferase value to which will be compared the values measured in the test wells to identify potential inhibitor or activator coumpounds. In wells A12 to D12, cells are grown in medium without TGFβ. The firefly luciferase value obtained with these points represents the ‘basal firefly luciferase activity’ and allows to control that the TGFβ induction is correct. In wells E12 to H12, cells are incubated in the presence of TGFβ with 500 μM CPO (Cyclopentenone, Sigma) which is a cell toxic compound. The toxicity is revealed by a decreased firefly and renilla luciferase activities (around 50% of those obtained in column 11). These points allows to control that the test is sensitive to toxic compounds.

12 to 18 hours later (day 3), the luciferase quantification procedure is launched. The following reactions are realized using reagents of the Dual Luciferase Assay Kit from Promega. Cells are washed and lysed with the addition of 10 μl of passive lysis buffer (Promega). After 15 to 30 mn of agitation, luciferase activities of the plates are read in a dual-injector luminometer (BMG lumistar). For this purpose, 50 μl of luciferase assay reagent and 50 μl of Stop & Glo buffer are injected sequentially to quantify the activities of both luciferases. Data are then processed and analysed using adequate software.

Description of an Inhibitor of CAGA-Mediated Transcription

Several thousands chemical compounds have been assayed in the automated high troughput transcriptional screen described above. The α-cyano4-hydroxy-3-ethoxy-5-phenylthiomethyl cinnamamide compound, called hereafter compound A, has been found to have an inhibitory effect on the TGFβ-induced firefly luciferase activities of both clones F89 and 1613 (with an IC₅₀ between 5 and 10 μM) but not on the renilla luciferase activities, and is given as example.

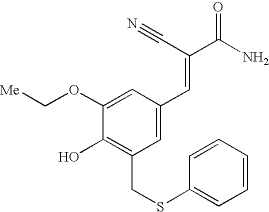

Compound A (α-Cyano4-Hydroxy-3-Ethoxy-5-Phenylthiomethyl Cinnamamide)

Table 4 shows the effect of increasing concentrations of compound A on the firefly luciferase activities of clones F89 and 1613 in the presence of 1 ng/mL of TGFβ (value 100 corresponds to the firefly luciferase activity observed in the absence of compound A and in the presence of 1 ng/mL TGFβ).

TABLE 4

| Compound A | | | | | |
|---|---|---|---|---|---|
| concentration (μM) | 0 | 0.1 | 1 | 5 | 10 |
| F 89 | 100 | 98 | 95 | 86 | 23 |
| 1613 | 100 | 105 | 102 | 65 | 36 |

1. A method for screening therapeutic agents for use in combating diseases associated with gene regulation by one or more Smad proteins and TGFβ or activin, said method comprising detecting or assaying the extent or result of transcriptional activity or binding in the presence of said agent between a Smad protein or a DNA binding fragment thereof and a double strand oligonucleotide comprising the sequence 5′ WXYCAGACZ 3′ or a functional equivalent thereof, wherein in said nucleotide sequence W represents A or G, X represents G or T, Y represents C, A, G or T and Z represents A or C.

2. A method according to claim 1 wherein the double strand oligonucleotide comprises the sequence 5′ WXYCAGACZ 3′ or a functional equivalent thereof, wherein in said nucleotide sequence W represents A or G, X represents G or T, Y represents C, A or G and Z represents A or C.

3. A method according to claim 1 or 2 wherein the double strand oligonucleotide comprises the sequence 5′ AG(C/A)CAGACA 3′,or a functional equivalent thereof.

4. A method according to claim 1 or 2 wherein the double strand oligonucleotide comprises the sequence 5′ ATGCAGACA 3′ or 5′ GGCCAGACA 3′, or a functional equivalent thereof.

5. A method according to any one of claims 1-3 for use in the treatment of fibrotic disorders, abnormal wound healing, abnormal bone formation, cancer development, haematopoiesis, neuroprotection and immune and inflammatory disorders.

6. A kit for screening agents suitable for combating diseases associated with gene regulation by one or more Smad proteins and TGFβ or activin, said kit comprising:
a Smad protein as nereinbefore defined
TGFβ or activin
a double strand DNA molecule comprising the sequence 5′ WXYCAGACZ 3′ or a functional equivalent thereof, wherein in said nucleotide sequence W represents A or G, X represents G or T, Y represents C, A or G and Z represents A or C, said sequence optionally being in operable linkage with a promoter or enhancer sequence and coding region of a gene whose product is detectable.

7. A method of treating a disease associated with gene regulation by means of one or more Smad proteins and TGFβ or activin, said method comprising administering to a mammal, including a human, a double strand oligonucleotide comprising the sequence 5′ WXYCAGACZ 3′ or a functional equivalent thereof, wherein in said nucleotide sequence W represents A or G, X represents G or T, Y represents C, A or G and Z represents A or C.

8. Use of a double strand oligonucleotide comprising the sequence 5′ WXYCAGACZ 3′ or a functional equivalent thereof, wherein in said nucleotide sequence W represents A or G, X represents G or T, Y represents C, A or G and Z represents A or C, in the treatment of a disease associated with gene regulation by one or more Smad proteins and TGFβ or activin.

9. Use of a double strand oligonucleotide comprising the sequence 5′ WXYCAGACZ 3′ or a functional equivalent thereof, wherein in said nucleotide sequence W represents A or G, X represents G or T, Y represents C, A or G and Z represents A or C, in the manufacture of a medicament for the treatment of a disease associated with gene regulation by one or more Smad proteins and TGFβ or activin.

10. A method of treating a disease associated with gene regulation by means of one or more Smad proteins and TGFβ or activin, said method comprising administering to a mammal, including a human, a therapeutic amount of an agent which inhibits or activates transcriptional activity or binding of said Smad proteins with a promoter or enhancer implicated in the gene regulation by TGFβ or activin, said promoter or enhancer comprising the nucleotide sequence 5′ WXYCAGACZ 3′ or a functional equivalent thereof, wherein in said nucleotide sequence W represents A or G, X represents G or T, Y represents C, A or G and Z represents A or C.

11. Use of a therapeutic amount of an agent which inhibits or activates transcriptional activity or binding of one or more Smad proteins with a promoter or enhancer implicated in the gene regulation by TGFβ or activin, said promoter or enhancer comprising the nucleotide sequence 5′ WXYCAGACZ 3′ or a functional equivalent thereof, wherein in said nucleotide sequence W represents A or G, X represents G or T, Y represents C, A or G and Z represents A or C, in the treatment of a disease associated with gene regulation by one or more Smad proteins and TGFβ or activin.

12. Use of a therapeutic amount of an agent which inhibits or activates transcriptional activity or binding of one or more Smad proteins with a promoter or enhancer implicated in the gene regulation by TGFβ or activin, said promoter or enhancer comprising the nucleotide sequence 5′ WXYCAGACZ 3′ or a functional equivalent thereof, wherein in said nucleotide sequence W represents A or G, X represents G or T, Y represents C, A or G and Z represents A or C, in the manufacture of a medicament for the treatment of a disease associated with gene regulation by one or more Smad proteins and TGFβ or activin.

13. A method of treating a disease associated with gene regulation by one or more Smad proteins and TGFβ or activin, comprising administration to a mammal, including a human, of a therapeutic amount of an agent identified in the method according to any one of claims 1-4.

14. Use of a therapeutic amount of an agent identified in the method according to any one of claims 1-4 in the treatment of a disease associated with gene regulation by one or more Smad proteins and TGFβ or activin.

15. Use of a therapeutic amount of an agent identified in the method according to any one of claims 1-4 in the manufacture of a medicament for the treatment of a disease with gene regulation by one or more Smad proteins and TGFβ or activin.

16. An isolated double strand DNA molecule comprising the sequence 5′ WXYCAGACZ 3′ or a functional equivalent thereof, wherein in said nucleotide sequence W represents A or G, X represents G or T, Y represents C, A, G or T and Z represents A or C.

17. An isolated double strand DNA molecule according to claim 16 which has the sequence 5′ AG(C/A)CAGACA 3′.

18. An isolated double strand DNA molecule according to claim 16 which has the sequence 5′ ATGCAGACA 3′.

19. An isolated double strand DNA molecule according to claim 16 which has the sequence 5′ GGCCAGACA 3′.

20. A therapeutic agent which inhibits or activates transcriptional activity or binding of one or more Smad proteins with a promoter or enhancer implicated in the gene regulation by TGFβ or activin, said promoter or enhancer comprising the nucleotide sequence 5′ WXYCAGACZ 3′ or a functional equivalent thereof, wherein in said nucleotide sequence W represents A or G, X represents G or T, Y represents C, A or G and Z represents A or C.

21. A therapeutic agent identified in a method according to any one of claims 1-4.
